(19) United States
(12) Patent Application Publication
(10) Pub. No.: US 2021/0283861 A1
(43) Pub. Date: Sep. 16, 2021

(54) APPARATUS AND METHOD FOR MANUFACTURING MICRONEEDLE

(71) Applicant: RESEARCH COOPERATION FOUNDATION OF YEUNGNAM UNIVERSITY, Gyeongsangbuk-do (KR)

(72) Inventors: Dong In LEE, Gyeongsangbuk-do (KR); Dong Su LEE, Seoul (KR); Bong Hwan KIM, Daegu (KR); Eun Ju KANG, Gyeonggi-do (KR)

(21) Appl. No.: 16/316,123

(22) PCT Filed: Jul. 10, 2017
(86) PCT No.: PCT/KR2017/007351

§ 371 Date: Jan. 8, 2019

(30) Foreign Application Priority Data
Jul. 13, 2016 (KR) 10-2016-0088388

(51) Int. Cl. B29C 67/00; A61M 37/00

(52) U.S. Cl. CPC B29C 67/0003; B29L 2031/7544; A61M 2037/0053; A61M 37/0015

(57) ABSTRACT

An apparatus for fabricating a microneedle includes a first substrate having a plurality of first spaced viscous materials formed on one side thereof and including at least one first transmission area, a second substrate disposed opposite to the first substrate, having a plurality of second spaced viscous materials formed on one side thereof facing the first substrate and including at least one second transmission area corresponding to the first transmission area, a light emitting unit arranged to correspond to the first transmission area on the other side of the first substrate and emitting light, and a light receiving unit arranged to correspond to the second transmission area on the other side of the second substrate. The microneedle manufacturing apparatus checks whether the first substrate and the second substrate are aligned according to whether the light receiving unit receives light emitted from the light emitting unit.

### TECHNICAL FIELD

The present invention relates to microneedles.

### BACKGROUND ART

Microneedle is a new technology that enables painless delivery of growth hormones, insulin, vaccines, protein treatments, and the like, which are difficult to orally administered, through the skin. Microneedle technology is applicable to various applications, such as high functional cosmetic materials, drugs, medicines and medical devices.

Conventionally, microneedles are fabricated by a molding technique. However, when the microneedles are manufactured using the molding technique, the microneedles may be damaged in the process of separating a patch having the microneedles formed thereon from the molds, or the fabricated microneedles have week strength. Hence, an attention has recently been paid to a drawing technique, which has overcome the limitations of the molding technique. In the drawing technique, microneedles are fabricated by drawing up and tensioning viscous materials. However, even in the drawing technique, it is difficult to precisely align positions of viscous materials on an upper plate and a lower plate.

### DISCLOSURE

### Technical Problem

Exemplary embodiments of the present invention are directed to providing an apparatus and method for fabricating microneedles, which can precisely align viscous materials on a first substrate and viscous materials on a second substrate and bring them into contact with each other.

### Technical Solution

According to one exemplary embodiment, there is provided an apparatus for fabricating microneedles by a drawing technique, the apparatus including: a first substrate having a plurality of spaced first viscous materials formed on one side thereof and including at least one first transmission area; a second substrate disposed opposite to the first substrate, having a plurality of spaced second viscous materials on one side thereof facing the first substrate, and including at least one second transmission area corresponding to the first transmission area; a light emitting unit disposed on the other side of the first substrate, corresponding to the first transmission area, and configured to emit light; and a light receiving unit disposed on the other side of the second substrate, corresponding to the second transmission area, wherein the apparatus for fabricating microneedles checks whether the first substrate and the second substrate are aligned with each other according to whether the light receiving unit receives light emitted from the light emitting unit.

The light emitting unit, the first transmission area, the second transmission area, and the light receiving unit may be arranged in a straight line.

Some of the plurality of first viscous materials may be formed on each of the at least one first transmission area on the one side of the first substrate and some of the plurality of second viscous materials may be formed on each of the at least one second transmission area on the one side of the second substrate.

The apparatus may further include: a first stage to which the first substrate is secured; a second stage to which the second substrate is secured; and a stage driving unit which is configured to move at least one of the first stage and the second stage so that the light receiving unit is placed at a position to receive the light emitted from the light emitting unit.

The apparatus may further include a control unit configured to control the stage driving unit, wherein, when the control unit receives a light receiving signal from the light receiving unit, the control unit controls the stage driving unit to bring the first viscous materials and the second viscous materials into contact with each other.

The first transmission area may be formed by filling a through-hole passing through the first substrate with a transparent material or a translucent material and the second transmission area may be formed by filling a through-hole passing through the second substrate with a transparent material or a translucent material.

The first transmission area may be formed of a material having thermal strain whose difference from thermal strain of the first substrate falls within a predetermined threshold range and the second transmission area may be formed of a material having thermal strain whose difference from thermal strain of the second substrate falls within a predetermined threshold range.

According to another exemplary embodiment, there is provided an apparatus for fabricating microneedles by a drawing technique, the apparatus including: a first stage to which a first substrate having a plurality of spaced first viscous materials formed on one side thereof and including at least one first transmission area is secured, wherein the first substrate is secured so that the one side having the first viscous materials formed thereon faces upward; a second stage which is arranged to be placed above the first stage and to which a second substrate having a plurality of spaced second viscous materials formed on one side thereof and including at least one second transmission area is secured, wherein the second substrate is secured so that the one side having the second viscous materials formed thereon faces the first substrate; a light emitting unit arranged on a surface of the first stage having the first substrate secured thereon, corresponding to the first transmission area, and configured to emit light; a light receiving unit arranged on a surface of the second stage having the second substrate secured thereon, corresponding to the second transmission area; and a stage driving unit which is configured to move at least one of the first stage and the second stage so that the light receiving unit is placed at a position to receive the light emitted from the light emitting unit.

According to one exemplary embodiment, there is provided a method of fabricating microneedles by a drawing technique, the method including: forming at least one first transmission area on a first substrate; forming at least one second transmission area on a second substrate corresponding to the first transmission area; forming a plurality of mutually spaced first viscous materials on one side of the first substrate; forming a plurality of mutually spaced second viscous materials on one side of the second substrate; securing the first substrate onto a first stage having at least one light emitting unit formed thereon; and securing the second substrate onto a second stage having at least one light receiving unit formed thereon corresponding to the light emitting unit.

The first transmission area may be formed by filling a through-hole passing through the first substrate with a transparent material or a translucent material and the second transmission area may be formed by filling a through-hole passing through the second substrate with a transparent material or a translucent material.

Some of the plurality of first viscous materials may be formed on each of the at least one first transmission area on the one side of the first substrate and some of the plurality of second viscous materials may be formed on each of the at least one second transmission area on the one side of the second substrate.

The method may further include, subsequent to the securing of the second substrate, placing the second stage above the first stage so that the second substrate faces the first substrate; checking whether the light receiving unit receives light emitted from the light emitting unit, and when the light receiving unit fails to receive the light emitted from the light emitting unit, moving at least one of the first stage and the second stage so that the light receiving unit receives the light emitted from the light emitting unit.

### ADVANTAGEOUS EFFECTS

According to an embodiment of the present invention, a first substrate and a second substrate are automatically aligned with each other through a light emitting unit formed below the first substrate and a light receiving unit formed above the second substrate, so that first viscous materials on the first substrate and second viscous materials on the second substrate are brought into contact with each other at precise positions, thereby improving yield in fabricating microneedles, and reducing fabricating time and costs.

### DESCRIPTION OF DRAWINGS

FIG. 1 is a diagram schematically illustrating an apparatus for fabricating a microneedle according to one embodiment of the present invention.

FIG. 2 is a flowchart illustrating a method of fabricating microneedles according to one embodiment of the present invention.

FIG. 3 is a block diagram illustrating an example of a computing environment including a computing device suitable to use in exemplary embodiments.

### MODE FOR INVENTION

Hereinafter, detailed embodiments of the present invention will be described with reference to the accompanying drawings. The following detailed description is provided for a more comprehensive understanding of methods, devices and/or systems described in this specification. However, the methods, devices, and/or systems are only examples, and the present invention is not limited thereto.

In the description of the present invention, detailed descriptions of related well-known functions that are determined to unnecessarily obscure the gist of the present invention will be omitted. Some terms described below are defined in consideration of functions in the present invention, and meanings thereof may vary depending on, for example, a user or operator's intention or custom. Therefore, the meanings of terms should be interpreted based on the scope throughout this specification. The terminology used in the detailed description is provided only to describe embodiments of the present invention and not for purposes of limitation. Unless the context clearly indicates otherwise, the singular forms include the plural forms. It should be understood that the terms “comprises” or “includes” specify some features, numbers, steps, operations, elements, and/or combinations thereof when used herein, but do not preclude the presence or possibility of one or more other features, numbers, steps, operations, elements, and/or combinations thereof in addition to the description.

Furthermore, relative terms such as “below,” “lower,” “above,” and “upper” may be used herein to describe one element's relationship to another element as illustrated in the accompanying drawings. Since elements in exemplary embodiments of the present invention may be positioned in various orientations, such relative terms are used for illustrative purpose and not for purpose of limitation.

In addition, it will be understood that, although the terms first, second, etc. may be used herein to describe various elements, these elements should not be limited by these terms. These terms are only used to distinguish one element from another. For example, a first portion could be termed a second portion, and, similarly, a second portion could be termed a first portion without departing from the teachings of the invention.

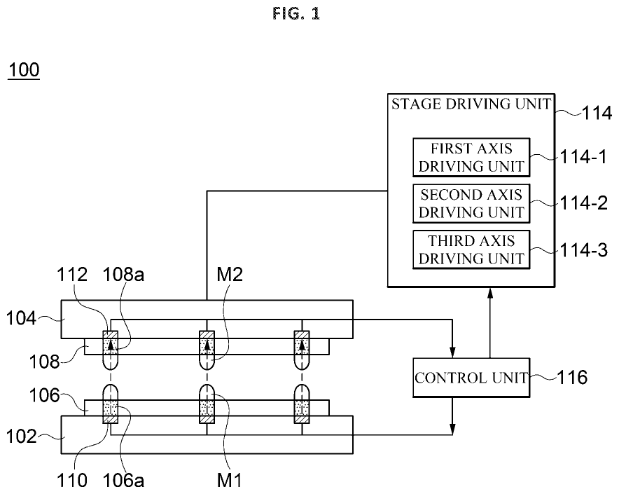

FIG. 1 is a diagram schematically illustrating an apparatus for fabricating a microneedle according to one embodiment of the present invention.

Referring to FIG. 1, the apparatus 100 for fabricating a microneedle may include a first stage 102, a second stage 104, a first substrate 106, a second substrate 108, a light emitting unit 110, a light receiving unit 112, a stage driving unit 114, and a control unit 116. The apparatus 100 for fabricating a microneedle may be an apparatus for fabricating microneedles by a drawing technique (i.e., a technique in which microneedles are fabricated by drawing up and tensioning viscous materials).

The first stage 102 may have an upper surface to which the first substrate 106 is seated and secured. The first stage 102 may be secured at a predetermined position. However, the first stage 102 is not limited to the above description and may be movably provided.

The second stage 104 may be positioned above the first stage 102. The second stage 104 may be located above and at a position corresponding to the first stage 102. The second stage 104 may have a lower surface to which the second substrate 108 is seated and secured.

The second stage 104 may be provided movably in a direction of a first axis (e.g., a widthwise direction of the second stage 104), a direction of a second axis (e.g., a longitudinal direction of the second stage 104), and a direction of a third axis (e.g., a thickness direction of the second stage 104).

The first substrate 106 may be secured onto the upper surface of the first stage 102. The first substrate 106 may be made of a substance, such as metal or ceramic, but is not limited thereto. A plurality of mutually spaced first viscous materials M1 for forming microneedles may be provided on an upper surface of the first substrate 106. The first substrate 106 may have at least one first transmission area 106a through which light emitted from the light emitting unit 110 can be transmitted. The first transmission area 106a may be formed of a transparent substance or a translucent substance. The first transmission area 106a may be formed of engineering plastic having thermal strain similar to that of the first substrate 106. That is, a difference between the thermal strain of the first transmission area 106a and the thermal strain of the first substrate 106 may fall within a preset threshold range. For example, the first transmission area 106a may be formed of a material having thermal strain whose difference from the thermal strain of the first substrate 106 is 10% or less.

Some of the plurality of first viscous materials M1 may be individually formed on the first transmission area 106a. The first transmission area 106a may have a size corresponding to a contact area between the first viscous materials M1 and the first substrate 106. In an exemplary embodiment, three first transmission areas 106a may be formed on the first substrate 106. In this case, the three first transmission areas 106a may be arranged in an equilateral triangular form on the first substrate 106. However, the embodiment is not limited to the above description, and more than three first transmission areas 106a may be provided and may be arranged in various forms, such as a polygon, a circle, an oval, or the like.

The second substrate 108 may be secured onto the lower surface of the second stage 104. The second substrate 108 may be formed of a material, such as metal or ceramic, but is not limited thereto. A plurality of spaced second viscous materials M2 for forming microneedles may be provided on a lower surface of the second substrate 108 (i.e., a surface facing the first substrate 106). The plurality of second viscous materials M2 may be provided to correspond to the plurality of first viscous materials M1, respectively.

The second substrate 108 may have at least one second transmission area 108a through which light emitted from the light emitting unit 110 can be transmitted. The second transmission area 108a may be formed of a transparent substance or a translucent substance. The second transmission area 108a may be formed of engineering plastic having thermal strain similar to that of the second substrate 108. That is, a difference between the thermal strain of the second transmission area 108a and the thermal strain of the second substrate 108 may fall within a preset threshold range. For example, the second transmission area 108a may be formed of a material having thermal strain whose difference from the thermal strain of the second substrate 108 is 10% or less. Some of the plurality of second viscous materials M2 may be formed on the second transmission area 108a.

The second transmission area 108a may be formed at a position corresponding to the first transmission area 106a. That is, the second transmission area 108a and the first transmission area 106a may be placed in a straight line in the third axis direction. In an exemplary embodiment, there may be three second transmission areas 108a formed on the second substrate 108 in the same manner as the first transmission areas 106a, and the three second transmission areas 108a may be arranged in an equilateral triangular form. However, the embodiment is not limited to the above description, more than three second transmission areas 108a may be provided and may be arranged in various forms, such as a polygon, a circle, an oval, or the like.

The light emitting unit 110 may be provided below the first transmission area 106a. The light emitting unit 110 may be provided below each of the first transmission areas 106a. In an exemplary embodiment, the light emitting unit 110 may be provided on the first stage 102 located on the lower side of the first substrate 106. The light emitting unit 110 may emit light in response to a light emission control signal of the control unit 116. The light emitting unit 110 may emit light rays of wavelengths of, for example, 630 nm to 1200 nm. In an exemplary embodiment, the light emitting unit 110 may be formed by a laser diode providing excellent linearity.

The light receiving unit 112 may be provided above the second transmission area 108a. The light receiving unit 112 may be provided above each of the second transmission area 118a. In an exemplary embodiment, the light receiving unit 112 may be formed on the second stage 104 located on the upper part of the second substrate 108. The light receiving unit 112 may receive light emitted from the light emitting unit 110. In this case, the light receiving unit 112 may generate an optical receiving signal to the control unit 116. The light receiving unit 112 and the light emitting unit 110 may be located in a straight line in the third axis direction.

Although herein the light receiving unit 110 is described as being located below the first transmission area 106a and the light receiving unit 112 is described as being located above the second transmission area 108a, the embodiment is not limited thereto such that the light emitting unit 110 may be located above the second transmission area 108a and the light receiving unit 112 may be located below the first transmission area 106a.

The stage driving unit 114 may be connected to the second stage 104. The stage driving unit 114 may move the second stage 104 in the first axis direction, the second axis direction, or the third axis direction under the control of the control unit 116. The stage driving unit 114 may include a first axis driving unit 114-a, a second axis driving unit 114-2, and a third axis driving unit 114-3. The first axis driving unit 114-1 may be provided to move the second stage 104 in the first axis direction. The second axis driving unit 114-2 may be provided to move the second stage 104 in the second axis direction. The third axis driving unit 114-3 may be provided to move the second stage 104 in the third axis direction.

The stage driving unit 114 may move the second stage 104 to be above the first stage 102, and then move the second stage 104 under the control of the control unit 116 such that the light receiving unit 112 (or the second transmission area 108a) is placed in a straight line with the light emitting unit 110 (or the first transmission area 106a). That is, the stage driving unit 114 may move the second stage 104 such that the light receiving unit 112 can receive light emitted from the light emitting unit 110. In this case, the first substrate 106 and the second substrate 108 are located at corresponding positions and vertically aligned with each other, the first viscous materials M1 of the first substrate 106 and the second viscous materials M2 of the second substrate 108 may be brought into contact with each other at precise positions.

In addition, the stage driving unit 114 may move the second stage 104 downward along the third axis direction to bring the first viscous materials M1 and the second viscous materials M2 into contact with each other. The stage driving unit 114 may move the second stage 104 upward in a state in which the first viscous materials M1 are in contact with the second viscous materials M2, and thereby may separate the first viscous materials M1 and the second viscous materials M2 from each other.

The control unit 116 may control the stage driving unit 114 so that the second stage 104 is located above the first stage 102. The control unit 116 may control the light emitting unit 110 to emit light by transmitting a light emission control signal to the light emitting unit 110. The control unit 116 may control the stage driving unit 114 to move the second stage 104, in response to a light receiving signal of the light receiving unit 112, so that the light receiving unit 112 (or the second transmission area 108a) can be located in a straight line with the light emitting unit 110 (or the first transmission area 106a).

According to an embodiment of the present invention, the first substrate 106 and the second substrate 108 are automatically aligned with each other through the light emitting unit 110 formed below the first substrate 106 and the light receiving unit 112 formed above the second substrate 108, so that the first viscous materials M1 of the first substrate 106 and the second viscous materials M2 of the second substrate 108 are brought into contact with each other at precise positions, thereby improving yield in fabricating microneedles, and reducing fabricating time and costs.

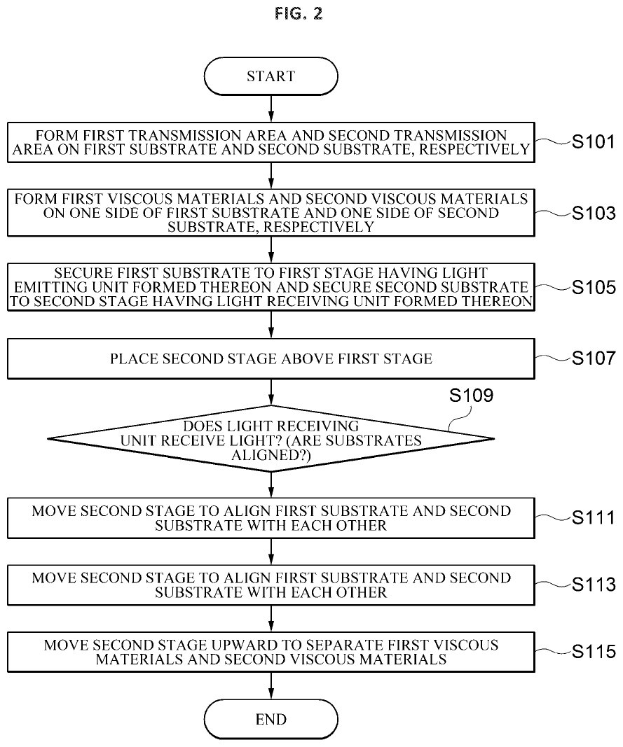

FIG. 2 is a flowchart illustrating a method of fabricating microneedles according to one embodiment of the present invention. The fabricating method may be performed by the above-described apparatus 100 for fabricating microneedles. Although in the illustrated flowchart the method is described as being divided into a plurality of operations, at least some of the operations may be performed in different order or may be combined into fewer operations or further divided into more operations. In addition, some of the operations may be omitted, or one or more extra operations, which are not illustrated, may be added to the flowchart and be performed.

Referring to FIG. 2, at least one first transmission area 106a is formed on the first substrate 106 and at least one second transmission area 108a is formed on the second substrate 108 (S101). For example, a through-hole of a predetermined cross-sectional area passing through each of the first substrate 106 and the second substrate 108 may be formed and then be filled with a transparent material or a translucent material, thereby forming the first transmission area 106a and the second transmission area 108a. In an exemplary embodiment, three first transmission areas 106a and three second transmission areas 108a may be arranged on the first substrate 106 and the second substrate 108, respectively, in an equilateral form. The first substrate 106 and the second substrate 108 may have the same size.

Then, the first viscous materials M1 are formed on one surface of the first substrate 106 and the second viscous materials M2 are formed on one surface of the second substrate 108 (S103). In this case, the first viscous materials M1 and the second viscous materials M2 may be formed such that they are placed at the same positions in their corresponding substrate, have the same spacing between each viscous material, and have the same size and the same number of viscous materials. In addition, some (e.g., three) of the plurality of first viscous materials M1 may be formed on the first transmission area 106a in the first substrate 106 and some (e.g., three) of the plurality of second viscous materials M2 may be formed on the second transmission area 108a in the second substrate 108.

Here, the first viscous materials M1 and the second viscous materials M2 may be formed of chemically inert materials, non-toxic to a human body. In addition, the first viscous materials M1 and the second viscous materials M2 may be formed of materials dissolvable in living body by body fluids, enzymes, microorganisms, and the like. The first viscous materials M1 and the second viscous materials M2 may be formed to have viscosity by being dissolved by suitable solvents. The composition materials of the first viscous materials M1 and the second viscous materials M2 and the method of forming the same are outside of the scope of the present invention, and thus detailed descriptions thereof will be omitted.

Then, the first substrate 106 is secured to the first stage 102 having the light emitting unit 110 formed thereon, and the second substrate 108 is secured to the second stage 104 having the light receiving unit 112 formed thereon (S105). In an exemplary embodiment, the light emitting unit 110 may be embedded in one surface of the first stage 102 and the light receiving unit 112 may be embedded in one surface of the second stage 104. A plurality of light emitting units 110 may be formed corresponding to the positions and the number of the first transmission areas 106a in the first stage 102. A plurality of light receiving units 112 may be formed corresponding to the positions and the number of the second transmission areas 108a in the second stage 104.

Then, the second stage 104 is placed above the first stage 102 (S107). Specifically, the second stage 104 may be moved above the first stage 102 by the stage driving unit 114. The second stage 104 may be placed at a predetermined distance above the first stage 102. In this case, the first substrate 106 of the first stage 102 and the second substrate 108 of the second stage 104 face to each other.

Then, whether light emitted from the light emitting unit 110 is received by the light receiving unit 112 is checked (S109). When the first substrate 106 and the second substrate 108 are aligned with each other (more specifically, when the light emitting unit 110 (or the first transmission area 106a) and the light receiving unit 112 (or the second transmission area 108a) are aligned with each other), the light emitted from the light emitting unit 110 is received by the light receiving unit 112. However, when the first substrate 106 and the second substrate 108 are not aligned with each other, the light emitted from the light emitting unit 110 is not received by the light receiving unit 112.

When it is checked in operation S109 that the light emitted from the light emitting unit 110 is not received by the light receiving unit 112, the second stage 104 is moved in the first axis direction and the second axis direction so that the light emitted from the light emitting unit 110 can be received by the light receiving unit 112 (i.e., the first substrate 106 and the second substrate 108 are aligned with each other) (S111). In this case, when there are a plurality of light emitting units and light receiving units 112, the second stage 104 may be moved in the first axis direction and the second axis direction until a predetermined number of the light receiving units 112 or more receive the light from the light emitting units 110.

Then, when the first substrate 106 and the second substrate 108 are aligned with each other, the second stage 104 is moved downward in the third axis direction so that the first viscous materials M1 of the first substrate 106 are brought into contact with the second viscous materials M2 of the second substrate 108 (S113). Since the first substrate 106 and the second substrate 108 are in an aligned state, the first viscous materials M1 and the second viscous materials M2 are brought into precise contact with each other at exactly corresponding positions.

Then, the second stage 104 is moved upward in the third axis direction after the elapse of a predetermined period of time so that the first viscous materials M1 and the second viscous materials M2 are separated from each other (S115). In this case, the predetermined period of time may be, for example, 5 to 10 minutes, but it may be set differently depending on the viscosity of the first viscous materials M1 and the second viscous materials M2. In the event where the second stage 104 is moved upward, the first viscous materials M1 and the second viscous materials M2 are tensioned and separated from each other so that microneedles are formed on each of the first substrate 106 and the second substrate 108.

Here, although the first substrate 106 and the second substrate 108 are described as being secured to the first stage 102 and the second stage 104, respectively, after the formation of the first viscous materials M1 and the second viscous materials M2 on the first substrate 106 and the second substrate 108, respectively, the embodiment is not limited thereto, and the first substrate 106 and the second substrate 108 may be secured to the first stage 102 and the second stage 104, respectively, and thereafter the first viscous materials M1 and the second viscous materials M2 may be formed on the first substrate 106 and the second substrate 108, respectively.

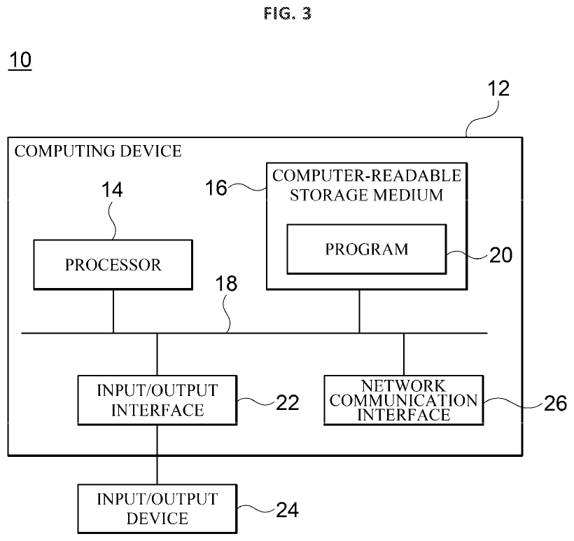

FIG. 3 is a block diagram illustrating an example of a computing environment 10 including a computing device suitable to use in exemplary embodiments. In the illustrated embodiment, each of the components may have functions and capabilities different from those described hereinafter and additional components may be included in addition to the components described herein.

The illustrated computing environment 10 includes a computing device 12. In one embodiment, the computing device 12 may be an apparatus for manufacturing microneedles (e.g., the apparatus 100 for manufacturing microneedles).

The computing device 12 includes at least one processor 14, a computer-readable storage medium 16, and a communication bus 18. The processor 14 may cause the computing device 12 to operate according to the aforementioned exemplary embodiment. For example, the processor 14 may execute one or more programs stored in the computer-readable storage medium 16. The one or more programs may include one or more computer executable commands, and the computer executable commands may be configured to, when executed by the processor 14, cause the computing device 12 to perform operations according to the exemplary embodiment.

The computer-readable storage medium 16 is configured to store computer executable commands and program codes, program data, and/or information in other suitable forms. The programs stored in the computer readable storage medium 16 may include a set of commands executable by the processor 14. In one embodiment, the computer-readable storage medium 16 may be a memory (volatile memory, such as random access memory (RAM), non-volatile memory, or a combination thereof) one or more magnetic disk storage devices, optical disk storage devices, flash memory devices, storage media in other forms capable of being accessed by the computing device 12 and storing desired information, or a combination thereof.

The communication bus 18 connects various other components of the computing device 12 including the processor 14 and the computer readable storage medium 16.

The computing device 12 may include one or more input/output interfaces 22 for one or more input/output devices 24 and one or more network communication interfaces 26. The input/output interface 22 and the network communication interface 26 are connected to the communication bus 18. The input/output device 24 may be connected to other components of the computing device 12 through the input/output interface 22. The illustrative input/output device 24 may be a pointing device (a mouse, a track pad, or the like), a keyboard, a touch input device (a touch pad, a touch screen, or the like), an input device, such as a voice or sound input device, various types of sensor devices, and/or a photographing device, and/or an output device, such as a display device, a printer, a speaker, and/or a network card. The illustrative input/output device 24 which is one component constituting the computing device 12 may be included inside the computing device 12 or may be configured as a separate device from the computing device 12 and connected to the computing device 12.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

1. An apparatus for fabricating microneedles by a drawing technique, the apparatus comprising:
a first substrate having a plurality of spaced first viscous materials formed on one side thereof and including at least one first transmission area;
a second substrate disposed opposite to the first substrate, having a plurality of spaced second viscous materials on one side thereof facing the first substrate, and including at least one second transmission area corresponding to the first transmission area;
a light emitting unit disposed on the other side of the first substrate, corresponding to the first transmission area, and configured to emit light; and
a light receiving unit disposed on the other side of the second substrate, corresponding to the second transmission area,
wherein the apparatus for fabricating microneedles checks whether the first substrate and the second substrate are aligned with each other according to whether the light receiving unit receives light emitted from the light emitting unit.

2. The apparatus of claim 1, wherein the light emitting unit, the first transmission area, the second transmission area, and the light receiving unit are arranged in a straight line.

3. The apparatus of claim 1, wherein some of the plurality of first viscous materials are formed on each of the at least one first transmission area on the one side of the first substrate and some of the plurality of second viscous materials are formed on each of the at least one second transmission area on the one side of the second substrate.

4. The apparatus of claim 1, further comprising:
a first stage to which the first substrate is secured;
a second stage to which the second substrate is secured; and
a stage driving unit which is configured to move at least one of the first stage and the second stage so that the light receiving unit is placed at a position to receive the light emitted from the light emitting unit.

5. The apparatus of claim 4, further comprising a control unit configured to control the stage driving unit,
wherein, when the control unit receives a light receiving signal from the light receiving unit, the control unit controls the stage driving unit to bring the first viscous materials and the second viscous materials into contact with each other.

6. The apparatus of claim 1, wherein the first transmission area is formed by filling a through-hole passing through the first substrate with a transparent material or a translucent material and the second transmission area is formed by filling a through-hole passing through the second substrate with a transparent material or a translucent material.

7. The apparatus of claim 1, wherein the first transmission area is formed of a material having thermal strain whose difference from thermal strain of the first substrate falls within a predetermined threshold range and the second transmission area is formed of a material having thermal strain whose difference from thermal strain of the second substrate falls within a predetermined threshold range.

8. An apparatus for fabricating microneedles by a drawing technique, the apparatus comprising:
a first stage to which a first substrate having a plurality of spaced first viscous materials formed on one side thereof and including at least one first transmission area is secured, wherein the first substrate is secured so that the one side having the first viscous materials formed thereon faces upward;
a second stage which is arranged to be placed above the first stage and to which a second substrate having a plurality of spaced second viscous materials formed on one side thereof and including at least one second transmission area is secured, wherein the second substrate is secured so that the one side having the second viscous materials formed thereon faces the first substrate;
a light emitting unit arranged on a surface of the first stage having the first substrate secured thereon, corresponding to the first transmission area, and configured to emit light;
a light receiving unit arranged on a surface of the second stage having the second substrate secured thereon, corresponding to the second transmission area; and
a stage driving unit which is configured to move at least one of the first stage and the second stage so that the light receiving unit is placed at a position to receive the light emitted from the light emitting unit.

9. A method of fabricating microneedles by a drawing technique, the method comprising:
forming at least one first transmission area on a first substrate;
forming at least one second transmission area on a second substrate corresponding to the first transmission area;
forming a plurality of mutually spaced first viscous materials on one side of the first substrate;
forming a plurality of mutually spaced second viscous materials on one side of the second substrate;
securing the first substrate onto a first stage having at least one light emitting unit formed thereon; and
securing the second substrate onto a second stage having at least one light receiving unit formed thereon corresponding to the light emitting unit.

10. The method of claim 9, wherein the first transmission area is formed by filling a through-hole passing through the first substrate with a transparent material or a translucent material and the second transmission area is formed by filling a through-hole passing through the second substrate with a transparent material or a translucent material.

11. The method of claim 9, wherein some of the plurality of first viscous materials are formed on each of the at least one first transmission area on the one side of the first substrate and some of the plurality of second viscous materials are formed on each of the at least one second transmission area on the one side of the second substrate.

12. The method of claim 9, further comprising: subsequent to the securing of the second substrate,
placing the second stage above the first stage so that the second substrate faces the first substrate;
checking whether the light receiving unit receives light emitted from the light emitting unit; and
when the light receiving unit fails to receive the light emitted from the light emitting unit, moving at least one of the first stage and the second stage so that the light receiving unit receives the light emitted from the light emitting unit.
